SEQUENCE LISTING (35 sequences)

SEQ ID NO: 1 - protein, 9 aa, Homo sapiens
KVLEYVIKV

SEQ ID NO: 2 - protein, 9 aa, Homo sapiens
FLWGPRALV

SEQ ID NO: 3 - protein, 9 aa, Homo sapiens
HLYQGCQVV

SEQ ID NO: 4 - protein, 10 aa, Homo sapiens
YLVPQQGFFC

SEQ ID NO: 5 - protein, 8 aa, Homo sapiens
APDTRPAP

SEQ ID NO: 6 - protein, 9 aa, Homo sapiens
NLTISDVSV

SEQ ID NO: 7 - protein, 10 aa, Human immunodeficiency virus type 1
PLTFGWCYKL

SEQ ID NO: 8 - protein, 9 aa, Human immunodeficiency virus type 1
SLYNTVATL

SEQ ID NO: 9 - protein, 9 aa, Human immunodeficiency virus type 1
TLNAWVKVV

SEQ ID NO: 10 - protein, 10 aa, Human immunodeficiency virus type 1
RGPGRAFVTI

SEQ ID NO: 11 - protein, 9 aa, Human immunodeficiency virus type 1
AFHHVAREL

SEQ ID NO: 12 - protein, 10 aa, Human immunodeficiency virus type 1
VLEWRFDSRL

SEQ ID NO: 13 - protein, 9 aa, Human immunodeficiency virus type 1
ILKEPVHGV

SEQ ID NO: 14 - protein, 9 aa, Human immunodeficiency virus type 1
VIYQYMDDL

SEQ ID NO: 15 - protein, 10 aa, Human immunodeficiency virus type 1
KYTAFTIPSI

SEQ ID NO: 16 - protein, 9 aa, Human immunodeficiency virus type 1
KLTPLCVTL

SEQ ID NO: 17 - protein, 9 aa, Human papillomavirus type 11
RLVTLKDIV

SEQ ID NO: 18 - protein, 10 aa, Human papillomavirus type 16
TIHDIILECV

SEQ ID NO: 19 - protein, 10 aa, Human papillomavirus type 16
YMLDLQPETT

SEQ ID NO: 20 - protein, 9 aa, Human papillomavirus type 16
LLMGTLGIV

SEQ ID NO: 21 - protein, 8 aa, Human papillomavirus type 16
TLGIVCPI

SEQ ID NO: 22 - RNA, 35 nt, Artificial Sequence
  RNA encoding peptide SIINFEKL
acaagaugaguauaaucaacuuugaaaaacuguaa

SEQ ID NO: 23 - RNA, 41 nt, Artificial Sequence
  RNA encoding peptide MFQEFVDWENV of mutated Kif18b
acaagauguuccaggaauuuguugacugggaaaacguuuaa

SEQ ID NO: 24 - RNA, 34 nt, Artificial Sequence
  RNA encoding peptide SIINFEKL
caagaugaguauaaucaacuuugaaaaacuguaa

SEQ ID NO: 25 - RNA, 27 nt, Artificial Sequence
  RNA encoding peptide SIINFEKL
augaguauaaucaacuuugaaaaacug

SEQ ID NO: 26 - RNA, 32 nt, Artificial Sequence
  RNA encoding peptide SIINFEKL
acaagaugaguauaaucaacuuugaaaaacug

SEQ ID NO: 27 - RNA, 24 nt, Artificial Sequence
  RNA encoding peptide SIINFEKL
aguauaaucaacuuugaaaaacug

SEQ ID NO: 28 - RNA, 30 nt, Artificial Sequence
  RNA encoding influenza virus epitope GILGFVFTL
auggggauuuugggguuuguguucacgcuc

SEQ ID NO: 29 - protein, 10 aa, Artificial Sequence
  Peptide comrising influenza virus M peptide GILGFVFTL
MGILGFVFTL

SEQ ID NO: 30 - protein, 9 aa, Influenza virus
GILGFVFTL

SEQ ID NO: 31 - RNA, 30 nt, Artificial Sequence
  RNA encoding peptide MNLVPMVATV
augaaccuggugcccaugguggcuacgguu

SEQ ID NO: 32 - protein, 10 aa, Artificial Sequence
  Peptide containing peptide NLVPMVATV of cytomegalo virus
MNLVPMVATV

SEQ ID NO: 33 - protein, 9 aa, cytomegalo virus
NLVPMVATV

SEQ ID NO: 34 - protein, 11 aa, Homo sapiens
MFQEFVDWENV

SEQ ID NO: 35 - RNA, 58 nt, Artificial Sequence
  RNA encoding peptide SIINFEKL
acaagauggagaguauaaucaacuuugaaaaacuguaaaaaaaaaaaaaaaaaaaaaa

(19) United States
(12) Patent Application Publication
(10) Pub. No.: US 2022/0307017 A1
(43) Pub. Date: Sep. 29, 2022

(54) Minimal Messenger RNAs and uses thereof

(71) Applicant: Universität Zürich, Zürich (CH)

(72) Inventor: Steve Pascolo, Zurich (CH)

(21) Appl. No.: 17/639,113

(22) PCT Filed: Aug. 28, 2020
(86) PCT No.: PCT/EP2020/074160

§ 371 Date: Feb. 28, 2022

(30) Foreign Application Priority Data
Aug. 29, 2019 (EP) 19194501.3
May 22, 2020 (EP) 20176153.3

(51) Int. Cl. C12N 15/11; A61P 35/00; A61K 39/00; A61K 35/17

(52) U.S. Cl. CPC C12N 15/11; A61P 35/00; A61K 39/0011; A61K 35/17; A61K 2039/53

(57) ABSTRACT

Chemically synthesized RNA molecules (useful for expression of a coding sequence have the general structure 5′-W-X-Y-(coding sequence)-Z-3′ wherein W is selected from the group consisting of a 5′-Cap, a free 5′-triphosphate group, a free 5′-disphosphate group, a free 5′-monophosphate group, a free 5′—OH group and chemically modified analogues of said 5′-Cap, said 5′-triphosphate group, said free 5′-disphosphate group or said free 5′-monophosphate group, X is an optional 5′UTR sequence, Y is an optional start codon, and Z is directly linked to the coding sequence and is selected from the group consisting of a free 3′—OH group, a stop codon and a stop codon linked, optionally via a 3′UTR sequence, to a poly(A) tail. RNA populations wherein at least 85% or more of the RNA population have the disclosed RNA and RNA populations containing the disclosed RNA wherein at least 1 % of a RNA is present being 1 nucleotide shorter in comparison to the full length RNA, express the amino acid sequence encoded by the coding sequence in a cell or organism, or in a cell-free expression system. Pharmaceutical compositions, vaccines and diagnostic tools comprising the RNA or the RNA populations are described.

### REFERENCE TO SEQUENCE LISTING

This application contains and incorporates by reference a Sequence Listing ASCII text file submitted via EFS-Web having the file name 34J3074.txt, which was created on Feb. 23, 2022, and is 6,467 bytes in size. This file includes no new matter from the computer readable format of the Sequence Listing submitted 4 Mar. 2021 for International Application No. PCT/EP2020/074160.

### FIELD OF THE INVENTION

The present invention relates to completely chemically synthesized RNA molecules (hereinafter also denoted as “ChemRNA”) which have a minimal structure useful for expression of a coding sequence. The ChemRNA of the invention has the general structure 5′-W-X-Y-(coding sequence)-Z-3′ wherein W is selected from the group consisting of a 5′-Cap, a free 5′-triphosphate group, a free 5′-disphosphate group, a free 5′-monophosphate group, a free 5′-OH group and chemically modified analogues of said 5′-Cap, said 5′-triphosphate group, said free 5′-disphosphate group or said free 5′-monophosphate group, X is an optional 5′UTR sequence, Y is an optional start codon, and Z is directly linked to the coding sequence and is selected from the group consisting of a free 3′-OH group, a stop codon and a stop codon linked, optionally via a 3′UTR sequence, to a poly(A) tail. The present invention further relates to RNA populations wherein at least 85% or more of the RNA population have the same chemical composition of a RNA of the invention and to RNA populations containing a RNA of the invention wherein at least 1% of a RNA is present being 1 nucleotide shorter in comparison to the full length RNA. The RNAs and RNA populations of the invention are of use for expressing the amino acid sequence encoded by the coding sequence in a cell or an organism, or in a cell-free expression system. The invention further relates to pharmaceutical compositions, vaccines as well as diagnostic tools comprising the RNA or the RNA populations.

### BACKGROUND OF THE INVENTION

Synthetic messenger RNA (mRNA) is being intensively developed as a vector for expressing proteins for vaccination (i.e. expression of antigens) and therapy, e.g. expression of proteins such as cytokines or antibodies, replacement of deficient or aberrant proteins in genetic diseases or repairing DNA using, e.g., CRISPR-CAS. According to the prior art, the mRNA is produced in vitro by enzymatic processes: typically, a template DNA is transcribed into RNA by a RNA polymerase (in vitro transcribed mRNA: ivt mRNA), then the DNA is degraded by a DNase and the mRNA is eventually polyadenylated by a poly-A-polymerase (Tusup et al. (2019) Chimia (Aarau) 73 (6), 391-394). The enzymatic production of mRNA is efficient, robust and allows the production of large amounts of therapeutic mRNA. However, it has two major shortcomings: (i) due to production through a biological process the resulting RNA is usually classified by regulatory authorities as a gene therapeutic product, and (ii) enzymatic production of mRNA usually requires the additional step of transcribing DNA into RNA making the process indirect, since also the DNA template needs to be produced beforehand. For purposes such as anti-cancer vaccines, particularly when the mRNA vaccine aims at triggering a T-cell immunity against mutations, the mRNA could be relatively short as it needs to encode only an epitope (which can be as short as 3 to 8 amino acids).

The technical problem underlying the present invention is to provide mRNAs overcoming the above problems encountered with enzymatically produced RNAs.

### SUMMARY OF THE INVENTION

The solution to the above technical problem is the provision of the embodiments of the present invention as defined in the claims as well as in the present description and the drawings.

In particular, the present invention provides a fully chemically synthesized RNA (also denoted herein as “ChemRNA”) having the structure of the following general formula (1):

5′-W-X-Y-(coding sequence)-Z-3′ (1)

wherein

W is selected from the group consisting of a 5′-Cap, a free 5′-triphosphate group, a free 5′-disphosphate group, a free 5′-monophosphate group, a free 5′-OH group and chemically modified analogues of said 5′-Cap, said 5′-triphosphate group, said free 5′-disphosphate group or said free 5′-monophosphate group;

X may or may not be present, and, if present is a 5′UTR sequence;

Y may or may not be present, and, if present is a start codon; and

Z is directly linked to the coding sequence and is selected from the group consisting of a free 3′-OH group, a stop codon and a stop codon linked, optionally via a 3′UTR, to a poly(A) tail.

Preferred ChemRNAs of the invention have one of the structures according to the following formulas (2) to (61):

5′-N7MeGppp-UTR-AUG-(coding sequence)-stop-polyA-3′ (2)

5′-N7MeGppp-UTR-AUG-(coding sequence)-stop-3′ (3)

5′-N7MeGppp-UTR-AUG-(coding sequence)-3′ (4)

5′-N7MeGppp-UTR-(coding sequence)-stop-polyA-3′ (5)

5′-N7MeGppp-UTR-(coding sequence)-stop-3′ (6)

5′-N7MeGppp-UTR-(coding sequence)-3′ (7)

5′-N7MeGppp-AUG-(coding sequence)-stop-polyA-3′ (8)

5′-N7MeGppp-AUG-(coding sequence)-stop-3′ (9)

5′-N7MeGppp-AUG-(coding sequence)-3′ (10)

5′-N7MeGppp-(coding sequence)-stop-polyA-3′ (11)

5′-N7MeGppp-(coding sequence)-stop-3′ (12)

5′-N7MeGppp-(coding sequence)-3′ (13)

5′-triP-UTR-AUG-(coding sequence)-stop-polyA-3′ (14)

5′-triP-UTR-AUG-(coding sequence)-stop-3′ (15)

5′-triP-UTR-AUG-(coding sequence)-3′ (16)

5′-triP-UTR-(coding sequence)-stop-polyA-3′ (17)

5′-triP-UTR-(coding sequence)-stop-3′ (18)

5′-triP-UTR-(coding sequence)-3′ (19)

5′-triP-AUG-(coding sequence)-stop-polyA-3′ (20)

5′-triP-AUG-(coding sequence)-stop-3′ (21)

5′-triP-AUG-(coding sequence)-3′ (22)

5′-triP-(coding sequence)-stop-polyA-3′ (23)

5′-triP-(coding sequence)-stop-3′ (24)

5′-triP-(coding sequence)-3′ (25)

5′-diP-UTR-AUG-(coding sequence)-stop-polyA-3′ (26)

5′-diP-UTR-AUG-(coding sequence)-stop-3′ (27)

5′-diP-UTR-AUG-(coding sequence)-3′ (28)

5′-diP-UTR-(coding sequence)-stop-polyA-3′ (29)

5′-diP-UTR-(coding sequence)-stop-3′ (30)

5′-diP-UTR-(coding sequence)-3′ (31)

5′-diP-AUG-(coding sequence)-stop-polyA-3′ (32)

5′-diP-AUG-(coding sequence)-stop-3′ (33)

5′-diP-AUG-(coding sequence)-3′ (34)

5′-diP-(coding sequence)-stop-polyA-3′ (35)

5′-diP-(coding sequence)-stop-3′ (36)

5′-diP-(coding sequence)-3′ (37)

5′-mP-UTR-AUG-(coding sequence)-stop-polyA-3′ (38)

5′-mP-UTR-AUG-(coding sequence)-stop-3′ (39)

5′-mP-UTR-AUG-(coding sequence)-3′ (40)

5′-mP-UTR-(coding sequence)-stop-polyA-3′ (41)

5′-mP-UTR-(coding sequence)-stop-3′ (42)

5′-mP-UTR-(coding sequence)-3′ (43)

5′-mP-AUG-(coding sequence)-stop-polyA-3′ (44)

5′-mP-AUG-(coding sequence)-stop-3′ (45)

5′-mP-AUG-(coding sequence)-3′ (46)

5′-mP-(coding sequence)-stop-polyA-3′ (47)

5′-mP-(coding sequence)-stop-3′ (48)

5′-mP-(coding sequence)-3′ (49)

5′-OH-UTR-AUG-(coding sequence)-stop-polyA-3′ (50)

5′-OH-UTR-AUG-(coding sequence)-stop-3′ (51)

5′-OH-UTR-AUG-(coding sequence)-3′ (52)

5′-OH-UTR-(coding sequence)-stop-polyA-3′ (53)

5′-OH-UTR-(coding sequence)-stop-3′ (54)

5′-OH-UTR-(coding sequence)-3′ (55)

5′-OH-AUG-(coding sequence)-stop-polyA-3′ (56)

5′-OH-AUG-(coding sequence)-stop-3′ (57)

5′-OH-AUG-(coding sequence)-3′ (58)

5′-OH-(coding sequence)-stop-polyA-3′ (59)

5′-OH-(coding sequence)-stop-3′ (60)

5′-OH-(coding sequence)-3′ (61)

wherein:

polyA is a poly(A) tail;

stop is a stop codon;

UTR is a 5′UTR

triP is a free triphosphate group;

diP is a free diphosphate group;

mP is a free monophosphate group.

As defined herein N7MeGppp is N7-methylguanosine triphosphate.

Specifically preferred is a ChemRNA of the invention according to formula (58).

Other particularly preferred ChemRNAs of the invention include those of formula (3)

In further preferred embodiments of the invention the ChemRNA is an RNA of formula (15).

In yet other preferred embodiments of the invention the ChemRNA has a structure according to formula (39).

In further preferred embodiments of the invention, the ChemRNA has a structure according to formula (51).

Yet in further preferred embodiments of the invention, the ChemRNA has a structure according to formula (61).

In one embodiment of the invention the RNA comprises a 5′-Cap, a 5′UTR, a start codon, a coding sequence and a stop codon as outlined in further preferred details in formula (3). Generally speaking such RNA of this embodiment of the invention can alternatively be defined by the following general structure:

5′-Cap-5′UTR-(start codon)-(coding sequence)-(stop codon)-3′

If present, the stop codon is preferably selected from UAA, UAG and UGA.

If present, the RNA of the invention preferably comprises a relatively short 5′UTR sequence. Particularly preferred 5′UTR sequences for use in the invention are selected from those 5′UTR sequences not exceeding 10 nucleotides (nt), more preferably 2 to 10 nt, i.e. the highly preferred 5′UTR sequences for use in the invention have a length of 2, 3, 4, 5, 6, 7, 8, 9, or 10 nt.

Examples of preferred 5′UTR sequences for use in the invention are, e.g. disclosed in Elfakess and Dikstein (2008) PLoS ONE 3 (8), e3094. Highly preferred 5′UTR sequences comprise the sequence 5′-AAG-3′. More particularly, 5′UTR sequences for the RNA of the invention comprise the motif 5′-AAG-3′ and have a length of 5 nt, wherein it is more preferred that the motif 5′-AAG-3′ directly precedes the start codon. A preferred 5′UTR sequence for use in the invention is the sequence 5′-ACAAG-3′. In other embodiments employing this motif, particularly preferred 5′UTR sequences of 5, 6, 7, or 8 nt, the 5′UTR can also comprise this sequence, wherein it is preferred that the 5 nt sequence 5′-ACAAG-3′ directly precedes the start codon.

In other embodiments of the invention the 5′UTR is selected from 5′UTR sequences disclosed in WO 2017/167910 A1. In particular, the 5′UTR preferably comprises or consists of, respectively the sequence 5′-CGCCACC-3′ wherein the C nucleotide at position 6 (counted from the 5′ end) may be substituted by an adenosine nucleotide and/or the C nucleotide at position 7 (counted from the 5′ end) may be substituted by a guanosine nucleotide and/or the A nucleotide at position 5 may be substituted by a guanosine nucleotide. Particularly preferred 5′UTR sequences of this type comprising such sequences are selected from those sequences where the sequence 5′-CGCCACC-3′ directly precedes the start codon. In other preferred embodiments, the 5′UTR comprises or consists of, respectively, the sequence 5′-CNGCCACC-3′ with N being selected from A, C, G and U, and wherein the C nucleotide at position 7 (counted from the 5′ end) may be substituted by an A nucleotide and/or the nucleotide at position 8 (counted from the 5′ end) may be substituted by a G nucleotide and/or the A nucleotide at position 6 (counted from the 5′ end) may be substituted by a G nucleotide. 5′UTR sequences of this type comprising such sequences are selected from those sequences where the sequence 5′-CNGCCACC-3′ directly precedes the start codon.

It is one of the surprising findings of the present invention that the RNAs disclosed and described herein useful for expression of the coding sequence do not need a 3′ poly(A) tail. Thus, preferred embodiments of RNA molecules disclosed herein do not contain a poly(A) tail at the 3′ end. In other embodiments of the invention the RNA contains a poly(A) tail at the 3′ end. If a poly(A) tail is present, it is preferably relatively short. Preferred poly(A) tails have up to 30 nt such as 2 to 30 nt, more preferably up to 20 nt such as 5 to 20 nt, even more preferred up 15 nt such as 5 to 15 nt, still further preferred up to 10 nt such as 5 to 10 nt. Particularly preferred lengths of poly(A) tails are 5, 10, 15, 20, 25, and 30 nt.

It is a further, and even more, surprising finding according to the invention that preferred embodiments of the ChemRNAs do not need a 5′-Cap structure for being useful in expression of the coding sequence, in particular in a cell or organism.

Moreover, it is yet a further highly surprising finding that the ChemRNA of the invention can also lack a phosphate group at the 5′ end (i.e. the 5′-end group is OH) for being useful in expression of the coding sequence.

It is a further highly surprising finding of the invention that ChemRNAs even do not need a start codon and/or a stop codon for being useful in expression of the coding sequence.

Furthermore, due to their completely chemical production process, RNAs and populations thereof according to the present invention may not considered as gene therapeutic product (cf. Hinz et al. (2017) Methods in Mol. Biol. 1499, 203-222) making regulatory approval procedures much easier and faster.

Preferred RNAs of the invention are RNA oligonucleotides. RNA oligonucleotides of the invention preferably have a length of (i.e. consist of) not more than 200 nt, more preferably the length is at most 100 nt, more preferably at most 80 nt, even more preferred at most 70 nt. Particularly preferred oligonucleotide RNAs of the invention have a length of from 24, 25, 26, 27, 28, 29 or 30 to 200 nt, more preferred from 24, 25, 26, 27, 28, 29 or 30 to 120 nt, still more preferred from 24, 25, 26, 27, 28, 29 or 30 to 100 nt.

It is also preferred that the RNA is single stranded. In other embodiments of the invention, the RNAs as defined and disclosed herein may also be partially or completely double stranded. Partially double stranded RNAs of the invention may contain only one strand forming double stranded parts or regions, or only one part or region, of double stranded structure due to self-complementary sequence sections in the single stranded RNA forming a hairpin. It is therefore to be understood that, in the case of partially double stranded RNAs of the invention resulting from self complementarity that such partially double stranded RNAs of the invention also are single stranded RNA. Other partially double stranded RNAs of the invention are composed of more than one, typically two strands having complementary sequence, whereby it is understood that, although formulas of RNAs of the invention show only one strand, the sequence of a strand being fully or partially complementary to the strand as shown in various embodiments herein is determined by the complementarity rules of RNA base pairing known in the art. The partially double stranded RNA of the invention formed by more than one, typically two, strands, can adopt any form such as staggered double strands, double stranded RNA having one blunt end and one end having an overhang, a double stranded RNA having two overhangs wherein the overhang are formed by the same strand etc. It is also contemplated according to the invention that double stranded RNAs are formed by more than two strands such as species wherein two strands are present being complementary to different regions of a third RNA strand. In certain embodiments of the invention, the RNA can also be completely double stranded having two blunt ends. In certain embodiments, double stranded RNAs, in particular those composed of more than one, preferably two, individual strands may serve, e.g. as precursors for providing a single strand encoding the peptide through the included coding sequence.

Fully or partially double stranded RNAs of the invention may also provide further functionalities to the RNA. In preferred embodiments, double stranded RNAs of the invention as defined above are contemplated having a free 5′ triphosphate being attached to one strand of a blunt end of a double stranded RNA of the invention such that it can function as a ligand of RIG-I. Other embodiments relate to RNAs capable of triggering TLRs such as double stranded RNAs of the invention having a length of 45 bp or more, typically 50 bp or more, triggering TLR3.

The RNA of the invention contains a coding sequence and is preferably useful for expressing the coding sequence in a cell in vitro or in vivo, or in a cell-free in vitro expression system. For the application in a cell-free expression system, RNAs of the invention having no 5′-Cap or first or second, respectively, RNA population containing such RNAs of the invention lacking a 5′-Cap are particularly preferred. According to the invention, the RNA as defined and disclosed herein is also referred to “coding RNA”. Although the RNA of the invention does not need to contain a 3′ poly(A) tail and/or a 5′-Cap and/or a start codon and/or a stop codon, the RNA of the present invention is also denoted as “mRNA”.

The coding sequence of the RNA molecules as disclosed herein is not specifically limited. Preferred coding sequences are selected such that the overall length of the RNA essentially complies with the overall length boundaries of RNA oligonucleotides as outlined before. Preferred coding sequences encode 4 to 65 amino acids. Particularly preferred coding sequences for use in the invention are relatively short, and encode 4 to 40 amino acids. More preferred the coding sequence encodes an amino acid sequence of 8 to 30 amino acids.

As further outlined in more detail below, preferred peptides encoded by the coding sequence are peptides, such as preferably epitopes, derived from cancer or tumor proteins (also denoted herein as “tumor-antigens”), or from infectious agents such as preferably viruses, bacteria or fungi.

Peptides derived from cancer or tumor, respectively, associated proteins, polypeptides or oligopeptides, respectively, are defined herein as “cancer peptides” and may have, in certain preferred embodiments, at least one amino acid that is different from the amino acid sequence of the non-cancer wildtype sequence.

Further preferred peptides encoded by the coding sequence contained in the RNA species of the invention are peptides of tissues recognized by autoimmune cells.

Another advantage of the present invention is the possibility to provide mRNAs having site-specific chemical modifications at precise nucleotide positions, which is typically impossible in the case of mRNAs prepared by enzymatic synthesis. For example, it becomes feasible to provide a single nucleotide with a specific chemical modification (be it at the phosphate backbone, the ribose or the base moiety). In preferred embodiments, the RNA has a chemical modification at a single nucleotide. Preferred chemical modifications are present at the 3′-terminal nucleotide and/or the 5′-terminal nucleotide.

Therefore, according to preferred embodiments of the invention, the RNA comprises at least one chemical modification, i.e. it comprises at least one chemically modified nucleotide analogue. In this context, a “medical modification” and “chemically modified nucleotide analogue” mean that the nucleotide is chemically modified in comparison to the corresponding canonical (i.e. unmodified) nucleotide a, c, g and u, respectively. The chemical modification may be at the phosphate, the ribose or the base moiety of the nucleotide. It is understood that, as used throughout the present specification, the term “nucleotide” refers to a “ribonucleotide”, if not specified otherwise. The modification(s) can be introduced during chemical synthesis or added on the ChemRNA by enzymes, for example from the families of methylases and deaminases. Another preferred example of an enzymatic modification is the addition of a poly(A) tail, preferably complying with the preferred length ranges as outlined above, to the 3′ end of the RNA, by incubation of a ChemRNA, preferably a ChemRNA having a structure according to formula (3), (6), (9), (12), (15), (18), (21), (24), (27), (30), (33), (36), (39), (42), (45), (48), (51), (54), (57) or (60), particularly preferred a ChemRNA having a structure according to formula (3), (15), (39) or (51), with a Poly(A) polymerase, such as Poly(A) polymerase from E. coli.

The chemical modification of the nucleotide analogue in comparison to the canonical nucleotide may be at the ribose, phosphate and/or base moiety. With respect to molecules having an increased stability, especially with respect to RNA degrading enzymes, modifications at the ribose and/or phosphate moieties, are especially preferred.

Preferred examples of ribose-modified ribonucleotides are analogues wherein the 2′-OH group is replaced by a group selected from H, OR, R, halo, SH, SR, NH₂, NHR, NR₂ or CN with R being C₁-C₆ alkyl, alkenyl or alkynyl and halo being F, Cl, Br or I. Highly preferred nucleotide analogues are methylated and fluorinated nucleotide analogues, most preferably 2′-O-methyl and 2′-F analogues.

As mentioned before, the at least one modified ribonucleotide may be selected from analogues having a chemical modification at the base moiety. Examples of such analogues include, but are not limited to, 5-aminoallyl-uridine, 6-aza-uridine, 8-aza-adenosine, 5-bromo-uridine, 7-deaza-adenosine, 7-deaza-guanosine, N⁶-methyl-adenosine, 5-methyl-cytidine, pseudo-uridine, N¹-methyl-pseudo-uridine, N¹-methyl-adenosine, thymine and 4-thio-uridine.

Examples of backbone-modified ribonucleotides wherein the phosphoester group between adjacent ribonucleotides is modified are phosphothioate groups.

Further preferred embodiments of RNAs according to the invention containing a modified nucleotide analogue are selected from RNAs wherein the modification is at the 3′ end of the RNA.

Preferred modifications include one of the modifications shown in the following table (left column: name of modified nucleotide analogue; right column: abbreviation) with the most preferred position of the respective nucleotide analogue being the 3′-terminus:
2′-O Methyl Adenosine A mA
2′-O Methyl Cytosine C mC
2′-O Methyl Guanosine G mG
2′-O Methyl Uridine U mU
2′-Fluoro deoxyadenosine (2′-F-A) 2-F-A
2′-Fluoro deoxycytosine (2′-F-C) 2-F-C
2′-Fluoro deoxyguanosine (2′-F-G) 2-F-G
2′-Fluoro deoxyuridine (2′-F-U) 2-F-U
propyne dC deoxycytosine pdC
propyne dU deoxyuridine pdU
L-DNA
L-RNA
Inverted dA (5′-5′ or 3′-3′ linkage) inv-dA
Inverted dC (5′-5′ or 3′-3′ linkage) inv-dC
Inverted dG (5′-5′ or 3′-3′ linkage) inv-dG
Inverted dT (5′-5′ or 3′-3′ linkage) inv-dT
Inverted rA (5-5′ or 3′-3′ linkage) rev-rA
Inverted rG (5-5′ or 3′-3′ linkage) rev-rC
Inverted rG (5-5′ or 3′-3′ linkage) rev-rG
Inverted rU (5-5′ or 3′-3′ linkage) rev-rU
Inverted 2′,3′ dideoxy dT (5′ Inverted ddT) ddT-5′
Phosphorothioate (PS) Bonds *
Methylphosphonate MP
Phosphorylation P
C3 Spacer SPC3

The RNA of the invention may also comprise chemical analogues of the 5′Cap or of the free 5′-phosphate group(s), namely, a free 5′-triphosphate, a free 5′-diphosphate or a 5′-monophosphate, as comprised in the definition of the group W according to formula (1). Typical, and preferred, analogues of the phosphate-containing 5′ groups are thiophosphates whereby preferred thiophosphates contain one sulfur atom per phosphate group. It is understood that those 5′ phosphate-containing groups which have more than one phosphate (i.e. a free 5′-diphosphate group, a free 5′-triphosphate group or a 5′Cap), may comprise more than one thiophosphate such as, preferably two thiophosphate moieties. The introduction of thiophosphates into 5′Cap and free 5′-phosphate group, respectively, is known in the art. For thiophosphate-containing 5′Cap structures it is referred e.g., to Strenkowska et al. (2016) Nucleic Acids Research 44 (20), pages 9578-9590.

Protocols for the chemical synthesis of RNAs of the invention is generally known in the art, and is typically carried by solid phase procedures based on the phosphoamidite method (see, for example, Beaucage and Iyer (1992) Tetrahedron Vol. 48. No. 12, pp. 2223-2311; Beaucage and Reese (2009) Curr. Protoc. Nucleic Acid Chem. 38:2.16.1-2.16.31).

Further subject matter of the invention is a (first) RNA population wherein at least 85%, preferably at least 90%, more preferably at least 95% of the RNAs in said population have the same chemical composition as a RNA as defined above, wherein the RNA may be understood to be defined as fully chemically synthesized or may be defined as outlined before, but without the explicit attribute of being “fully chemically synthesized”.

Another aspect of the invention is a further (second) RNA population comprising a RNA as defined herein above, which RNA has a full length of n nt and at least 1% of a RNA having a chemical composition being at least 95%, preferably at least 96%, more preferably at least 97%, still more preferred at least 98%, even more preferred at least 99% identical to the chemical composition of the full length RNA but having a length of (n−1) nt wherein the percentage of identity of the chemical composition of the RNA of length (n−1) to the chemical composition of the full length RNA of length is meant with respect to the chemical composition of the (n−1) nucleotides of the full length RNA of length n (i.e. the RNA having (n−1) nt present in an amount of at least 1% is one nucleotide shorter in comparison to the full-length RNA of length n but otherwise the nucleotide sequence is at least 95%, preferably at least 96%, more preferably at least 97%, still more preferred at least 98%, even more preferred at least 99% identical to the nucleotide sequence of the full-length RNA of length n). In a further preferred embodiment, this RNA population further contains at least 1% of a RNA having a chemical composition being at least 93%, preferably at least 95%, more preferably at least 96%, even more preferred at least 97%, still more preferred at least 98%, particularly preferred at least 99% identical to the chemical composition of the full length RNA the full length RNA but having a length of (n−2) wherein the percentage of identity of the chemical composition of the RNA of length (n−2) to the chemical composition of the full length RNA of length is meant with respect to the chemical composition of the (n−2) nucleotides of the full length RNA of length n (i.e. the RNA having (n−2) nt present in an amount of at least 1% is two nucleotides shorter in comparison to the full-length RNA of length n but otherwise the nucleotide sequence is at least 93%, preferably at least 95%, more preferably at least 96%, even more preferred at least 97%, still more preferred at least 98%, particularly preferred at least 99% identical to the nucleotide sequence of the full-length RNA of length n). In a still further preferred embodiment, the RNA population further contains at least 1% of a RNA having a chemical composition being at least 93%, preferably at least 95%, more preferably at least 96%, even more preferred at least 97%, still more preferred at least 98%, particularly preferred at least 99% identical to the chemical composition of the full length RNA as the full length RNA but having a length of (n−3) wherein the percentage of identity of the chemical composition of the RNA of length (n−3) to the chemical composition of the full length RNA of length is meant with respect to the chemical composition of the (n−3) nucleotides of the full length RNA of length n (i.e. the RNA having (n−3) nt present in an amount of at least 1% is one nucleotide shorter in comparison to the full-length RNA of length n but otherwise the nucleotide sequence is at least 90%, preferably at least 95%, more preferably preferably at least 96%, even more preferred at least 97%, still more preferred at least 98%, particularly preferred at least 98.5% identical to the nucleotide sequence of the full-length RNA of length n). Also in this embodiment of the invention the RNA may be understood to be defined as fully chemically synthesized or may be defined as outlined before, but without the explicit attribute of being “fully chemically synthesized”. According to the invention all references with respect to “n” concerning the second RNA population as disclosed herein are understood that “n” is an integer, such as an integer of at least 10, in certain embodiments of the invention at least 20, in other preferred embodiments of the invention at least 30 preferably of from 20 to 200, more preferred from 30 to 200, even more preferred from 30 to 120, still more preferred from 30 to 100.

The present invention is also directed to a pharmaceutical composition comprising a RNA as defined herein or a first RNA population as defined herein or a second RNA population as defined herein, optionally in combination with one or more pharmaceutically acceptable carrier(s), excipient(s) and/or diluent(s). Preferably, the pharmaceutical composition is in the form of a vaccine comprising an RNA as defined herein or a first RNA population as defined herein or a second RNA population as defined herein.

To further increase effectiveness, the vaccine according to the invention preferably comprises one or more adjuvants, preferably to achieve a synergistic effect of vaccination. “Adjuvant” in this context encompasses any compound which promotes an immune response. Various mechanisms are possible in this respect, depending on the various types of adjuvants. For example, compounds which allow the maturation of the DC, e.g. lipopolysaccharides or CD40 ligand, form a first class of suitable adjuvants. Generally, any agent which influences the immune system of the type of a “danger signal” (LPS, GP96, dsRNA etc.) or cytokines, such as GM-CSF, can be used as an adjuvant which enables an immune response to be intensified and/or influenced in a controlled manner. CpG oligodeoxynucleotides can optionally also be used in this context, although their side effects which occur under certain circumstances are to be considered. Particularly preferred adjuvants are cytokines, such as monokines, lymphokines, interleukins or chemokines, e.g. IL-1, IL-2, IL-3, IL-4, IL-5, IL-6, IL-7, IL-8, IL-9, IL-10, IL-12, INFα, INF-γ, GM-CFS, LT-α, or growth factors, e.g. hGH. Further known adjuvants are aluminium hydroxide, Freund's adjuvant or oil such as Montanide®, most preferred Montanide® ISA51. Lipopeptides, such as Pam3Cys, are also particularly suitable for use as adjuvants in the vaccine and/or pharmaceutical composition of the present invention.

In a preferred embodiment, the vaccine according to the invention can also be used in conjunction with another therapeutic reagent. The vaccine of the present invention may synergize with other treatments such as chemotherapeutic drugs for cancer patients, immune checkpoint inhibitors or tri-therapy for HIV patients or chloroquine, a drug used against malaria infection and known to improve cross priming.

The vaccine composition of the present invention is used in genetic vaccination, wherein an immune response is stimulated by introduction into the organism, wherein the RNA may be applied in naked form (i.e., in particular, uncomplexed form) or included in particles such as in complex with cationic ions, liposomes or polymers, or into the cell (for example, by in vitro electroporation followed by adoptive transfer or direct injection by needle-dependent or needle-less devices) a RNA or a first or second RNA population as disclosed herein. The vaccine composition of the invention can be injected systematically, preferably by intra-venous or sub-cutaneous injection, as well as locally at the site of the required mRNA delivery such as injection into a tumor, a muscle, the dermis or into a lymph node. Other preferred administration routes are intranasal administration and oral administration. In an alternative embodiment, antigen presenting cells such as DCs (or a progenitor cell population like PBMCs from which DCs are first isolated or at least enriched) from a patient to be treated are prepared (typically from a blood sample taken from the patient) into which RNA of the invention or a RNA population of the invention is introduced. Optionally, after an incubation step, the RNA-loaded DCs are re-introduced into the patient, preferably by intra venous administration.

The vaccines according to the invention are suitable for the treatment of cancers and tumors. Preferably, the RNA of the present invention, or the RNAs in the first or second RNA populations of the invention comprise(s) a coding sequence encoding an epitope of a tumor-specific antigen (TSA). Specific examples of tumor antigens from which epitopes to be encoded by the RNA/RNA population are derived include 707-AP, AFP, ART-4, BAGE, .beta.-catenin/m, Bcr-abl, CAMEL, CAP-1, CASP-8, CDC27/m, CDK4/m, CEA, CT, Cyp-B, DAM, ELF2M, ETV6-AML1, G250, GAGE, GnT-V, Gp100, HAGE, HER-2/neu, HLA-A*0201-R1701, HPV-E7, HSP70-2M, HAST-2, hTERT (or hTRT), iCE, KIAA0205, LAGE, LDLR/FUT, MAGE, MART-1/Melan-A, MC1R, myosin/m, MUC1, MUM-1, -2, -3, NA88-A, NY-ESO-1, p190 minor bcr-abl, Pml/RAR.alpha., PRAME, PSA, PSM, RAGE, RU1 or RU2, SAGE, SART-1 orSART-3, TEL/AML1, TPI/m, TRP-1, TRP-2, TRP-2/INT2 and WT1. With respect to specific sequences of MHC associated epitopes derived from tumor antigens it is referred to https://syfpeithi.de. Particularly preferred coding sequences in the RNA of the invention encode HLA-A*02:01-associated epitopes, more specifically KVLEYVIKV (SEQ ID NO: 1) from MAGE-A1, FLWGPRALV (SEQ ID NO: 2) from MAGE-A3, HLYQGCQVV (SEQ ID NO: 3) and YLVPQQGFFC (SEQ ID NO: 4) from HER-2/neu, APDTRPAP (SEQ ID NO: 5) and/or NLTISDVSV (SEQ ID NO: 6) from MUC1. In other preferred embodiments of the invention the coding sequence of the RNA encodes a tumor epitope containing one or more mutations found in a tumor. Specific and examples of preferred tumor epitopes of this kind are, e.g. enclosed in Sahin et al. (2017) Nature 547, 222-226, and more specifically to the epitopes found in the columns named “AA sequence”, “Predicted MHC I epitope” and “Predicted MHC II epitope”, respectively, of Supplementary Table 1 and in column “Amino acid sequence” of Supplementary Table 2 of this publication, to which sequences it is herein explicitly referred. Cancer peptides can be also for example epitopes from the hypervariable loops of TCR or immunoglobulin chains, in particular those being specific of clonotypic lymphoma or leukemia cells.

The vaccine according to the invention may be furthermore employed against infectious diseases. Preferred epitopes to be encoded by the coding sequences of the embodiments of the invention are contained in the infectious agents causing: AIDS (HIV), hepatitis A, B or C, herpes, herpes zoster (chicken-pox), German measles (rubella virus), yellow fever, dengue etc. flaviviruses, influenza viruses, coronaviruses, hemorrhagic infectious diseases (Marburg or Ebola viruses), bacterial infectious diseases, such as Legionnaire's disease (Legionella), gastric ulcer (Helicobacter), cholera (Vibrio), infections by E. coli, Staphylococci, Salmonella or Streptococci (tetanus); infections by protozoan pathogens such as malaria, sleeping sickness, leishmaniasis; toxoplasmosis, i.e. infections by Plasmodium, Trypanosoma, Leishmania and Toxoplasma, respectively; or fungal infections such as fungal infections which are caused e.g. by Cryptococcus neoformans, Histoplasma capsulatum, Coccidioides immitis, Blastomyces dermatitidis or Candida albicans). Preferred embodiments of the inventive RNA encode HLA-A*02:01-presented epitopes from such pathogens are, for example: HIV-1-derived epitopes preferably selected from PLTFGWCYKL (SEQ ID NO: 7), SLYNTVATL (SEQ ID NO: 8), TLNAWVKVV (SEQ ID NO: 9), RGPGRAFVTI (SEQ ID NO: 10), AFHHVAREL (SEQ ID NO: 11), VLEWRFDSRL (SEQ ID NO: 12), ILKEPVHGV (SEQ ID NO: 13), VIYQYMDDL (SEQ ID NO: 14), KYTAFTIPSI (SEQ ID NO: 15) and KLTPLCVTL (SEQ ID NO: 16) or epitopes derived from HPV11 preferably, e.g., RLVTLKDIV (SEQ ID NO: 17) or epitopes derived from HPV16 preferably selected from TIHDIILECV (SEQ ID NO: 18), YMLDLQPETT (SEQ ID NO: 19), LLMGTLGIV (SEQ ID NO: 20) or TLGIVCPI (SEQ ID NO: 21). Further examples of preferred epitopes include epitopes of influenza viruses, more preferably influenza A and B subtypes, particularly epitopes derived from influenza A, and coronaviruses, more preferably epitopes derived from SARS-CoV-1, SARS-CoV-2 and MERS-CoV. A preferred example of a peptide, more preferably an epitope of pathogenic bacteria is a peptide, more preferably an epitope, of Mycobacterium tubercolosis. As in the case of tumor antigens, many specific sequences of epitopes to be encoded by the coding sequences of the RNA according to the invention are known to the skilled person and may be selected from the database available at https://syfpeithi.de.

For all specific epitopes as disclosed explicitly herein as well as disclosed by way of reference to publications and public epitope databases, respectively, it is understood that, according to certain embodiments, the coding sequence of the RNA of the invention can encode a sequence comprising a specific epitope sequence, in particular a specific MHC class I epitope sequence or a specific MHC class II epitope sequence. In other embodiments, the coding sequence of the RNA according to the invention consists of a nucleotide sequence encoding such specific epitope.

The vaccine according to the invention may be used in combination with chloroquine, a pharmaceutical compound that increases cross presentation and thus the induction of antigen-specific effector T-cells.

The embodiments of the invention, in particular the RNA, the first RNA population and the second RNA population are useful as medicaments. The embodiments of the invention, in particular the RNA, the first RNA population and the second RNA population are particularly useful in the treatment of cancer and tumors, and also in the treatment and/or prevention of infectious diseases such infections by viral, prokaryotic and fungal infectious agents.

The invention also provides the use of the RNA and/or the first RNA population and/or the second RNA population as disclosed herein for the preparation of a medicament for the treatment of cancer and tumors. The invention also provides the use of the RNA and/or the first RNA population and/or the second RNA population as disclosed herein for the preparation of a medicament for the treatment and/or prevention of infectious diseases.

The invention furthermore provides a method of treating cancer or a tumor in a subject comprising administering to the subject in need thereof an effective amount of a pharmaceutical composition according to the invention.

The invention furthermore provides a method of treating and/or preventing an infectious disease in a subject comprising administering to the subject in need thereof an effective amount of a vaccine according to the invention.

Further subject matter of the invention is a diagnostic kit comprising at least one RNA and/or a first and/or a second RNA population of the invention. Preferably, the RNA or RNAs, respectively, encode(s) a peptide of an infectious agent such as preferably a peptide of a virus, a bacterium or a fungus. Preferred peptides are epitopes of such infectious agents. Examples of specific, and preferred, epitopes are outlined above with respect to the vaccine of the invention.

The diagnostic kit preferably further contains at least one transfection reagent, such as, e.g. a liposome reagent, and/or equipment or equipment parts for carrying out detection and/or separation methods (e.g. electrodes for electroporation).

The invention further relates to a method for diagnosis of a cancer, an autoimmune disease, an infectious disease and/or the presence of an infectious agent causing such a disease in a subject suspected of having said disease and/or being infected by the infectious agent comprising the steps of simulating a T cell population of the subject with at least one RNA and/or at least one first RNA population and/or at least one second RNA population comprising a coding sequence encoding a peptide, preferably an epitope, of said cancer, targeted tissue from autoimmune disease or infectious agent, and detecting the presence of T cells specific for said peptide, preferably said epitope. In the context of the invention a “T cell population” is a cell population of the subject comprising T cells. A typical T cell population is PBMCs obtained from the subject.

The step of stimulating the T cells preferably comprises the step of transfecting a cell population of the subject with at least one RNA and/or at least one first RNA population and/or at least one second RNA population comprising a coding sequence encoding a peptide, preferably an epitope, of said infectious agent, and detecting the presence of T cells specific for said peptide, preferably said epitope. After transfection, the cells are typically incubated under appropriate conditions for a time period of preferably 1 to 30.

The detection of the stimulated T cells typically involves the FACS analysis of the culture in a known fashion, preferably for CD3+CD4+ or CD3+CD8+ T cells specific for the antigen to be detected. Alternatively, secretion of cytokines from T-cells can be used to evaluate whether they are stimulated by the peptide encoded by the ChemRNA (ELISA or ELISpot to measure, for example, interleukine-2 (IL-2) or interferon-gamma (IFN-gamma) production).

The stimulation of T cells specific for a certain antigen, preferably a tumor or cancer antigen, can also be used in methods (and uses of the RNAs or populations of RNA according to the invention) for the treatment of tumors and cancer as already mentioned. In certain embodiments, T cells, i.e. typically a T cell population as described above, obtained from a subject suffering from cancer or tumor are transfected with an appropriate cancer peptide, detection and enrichment of the positive T cells, preferably by FACS, and back injection of the enriched anticancer peptide-stimulated T cells into the subject suffering from the cancer or tumor disease. Preferably, the detected and enriched T cells are expanded before being re-injected into the subject. Appropriate expansion techniques are known in the art. The method described above can also be used to stimulate specifically regulatory T-cells (Tregs) that can be used to control autoimmune diseases.

With respect to all and any disclosed, defined and described applications, uses and methods employing the RNAs having a structure as defined herein, the present invention is also directed to such applications, uses and methods wherein the RNA is an enzymatically synthesized RNA having the identical or essentially identical structure as the above defined fully chemically synthesized RNA, with the exception that the RNA is fully or substantially enzymatically prepared. Methods for enzymatic synthesis of RNA are known in the art. Typically an RNA polymerase such as T7 or Sp6 RNA polymerase are employed and various protocols including reagents as kits are commercially available from various suppliers (e.g., New England Biolabs Inc., Ipswich, Mass., USA; Promega Corp., Madison, Wis., USA; and various others).

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown.

FIG. 1 shows graphical representations of IL-2 release by OT1 mouse splenocytes alone (FIG. 1A) or OT1 mouse splenocytes plus B16 cells (FIG. 1B) transfected with the indicated agents after 18 hours of incubation as measured in the cell supernatant.

FIG. 2 shows graphical representations of IFN-gamma release by OT1 mouse splenocytes alone (FIG. 2A) or OT1 mouse splenocytes plus B16 cells (FIG. 2B) transfected with the indicated agents after 18 hours of incubation as measured in the cell supernatant.

FIG. 3 shows graphical representations of IL-2 release by OT1 mouse splenocytes transfected with the indicated agents after 18 hours of incubation as measured in the cell supernatant wherein FIG. 3A shows the results obtained with untreated RNAs and FIG. 3B shows the results obtained with enzymatically polyadenylated RNAs. The reagents were as follows: Capped 5n SIINFEKL: 5′-N7-MeGppp, 7mGppp-acaagAUGaguauaaucaacuuugaaaaacugUAA-3′ (SEQ ID NO: 22); ppp 5n SIINFEKL: 5′-ppp-acaagAUGaguauaaucaacuuugaaaaacugUAA-3′ (SEQ ID NO: 22); p 5n SIINFEKL: 5′-p-acaagAUGaguauaaucaacuuugaaaaacugUAA-3′ (SEQ ID NO: 22); OH 5n SIINFEKL: 5′-OH-acaagAUGaguauaaucaacuuugaaaaacugUAA-3′ (SEQ ID NO: 22); Kif18b capped oligo: 5′-N7-MeGppp, 7mGppp-acaagAUGuuccaggaauuuguugacugggaaaacguuUAA-3′ (SEQ ID NO: 23).

FIG. 4 shows a graphical representations of IL-2 release by OT1 mouse splenocytes transfected with the indicated agents after 44 hours of incubation as measured in the cell supernatant. The reagents were as follows: Cap 5n UTR: 5′-N7-MeGppp, 7mGppp-acaagAUGaguauaaucaacuuugaaaaacugUAA-3′ (SEQ ID NO: 22); 3P 5n UTR: 5′-ppp-acaagAUGaguauaaucaacuuugaaaaacugUAA-3′ (SEQ ID NO: 22); P 5n UTR: 5′-p-acaagAUGaguauaaucaacuuugaaaaacugUAA-3′; OH 5n UTR: 5′-OH-caagAUGaguauaaucaacuuugaaaaacugUAA-3′ (SEQ ID NO: 24); Min SIINFEKL: 5′-AUGAGUAUAAUCAACUUUGAAAAACUG-3′ (SEQ ID NO: 25); No STOP: 5′-ACAAGAUGAGUAUAAUCAACUUUGAAAAACUG-3′ (SEQ ID NO: 26); No AUG: 5′-AGUAUAAUCAACUUUGAAAAACUG-3′ (SEQ ID NO: 27); Kif18b capped oligo: 5′ N7-MeGppp, 7mGppp acaagAUGuuccaggaauuuguugacugggaaaacguuUAA 3′ (SEQ ID NO: 23).

FIG. 5 shows graphical representations of IL-2 release by OT1 mouse splenocytes transfected with the indicated agents after 24 hours of incubation as measured in the cell supernatant. The reagents were as follows: Cap 5n UTR: 5′-N7-MeGppp, 7mGppp-acaagAUGaguauaaucaacuuugaaaaacugUAA-3′ (SEQ ID NO: 22); 3P 5n UTR: 5′-ppp-acaagAUGaguauaaucaacuuugaaaaacugUAA-3′ (SEQ ID NO: 22); P 5n UTR: 5′-p-acaagAUGaguauaaucaacuuugaaaaacugUAA-3′ (SEQ ID NO: 22); OH 5n UTR: 5′-OH-caagAUGaguauaaucaacuuugaaaaacugUAA-3′ (SEQ ID NO: 24); Min SIINFEKL: 5′-AUGAGUAUAAUCAACUUUGAAAAACUG-3′ (SEQ ID NO: 25); No STOP: 5′-ACAAGAUGAGUAUAAUCAACUUUGAAAAACUG-3′ (SEQ ID NO: 26); No AUG: 5′-AGUAUAAUCAACUUUGAAAAACUG-3′ (SEQ ID NO: 27); Kif18b capped oligo: 5′-N7-MeGppp, 7mGppp-acaagAUGuuccaggaauuuguugacugggaaaacguuUAA-3′ (SEQ ID NO: 23).

FIG. 6 shows graphical representations of FACS analyses of PBMC cultures of a healthy donor after 7 days of incubation following no transfection of RNA (FIG. 6A), transfection with the RNA Oligo Flu matrix (5′OH-AUGGGGAUUUUGGGGUUUGUGUUCACGCUC-3′; SEQ ID NO: 28) encoding the influenza virus epitope GILGFVFTL (SEQ ID NO: 30) preceded by a methionine (FIG. 6B), and transfection with the RNA Oligo CMV pp65 (5-OH AUGAACCUGGUGCCCAUGGUGGCUACGGUU-3′; SEQ ID NO: 31) encoding the CMV epitope NLVPMVATV (SEQ ID NO: 33) preceded by a methionine.

FIG. 7 shows graphical representations of FACS analyses of PBMC cultures of a healthy donor after 14 days of incubation following no transfection with RNA (FIG. 7A), transfection with the RNA Oligo Flu matrix (5′OH-AUGGGGAUUUUGGGGUUUGUGUUCACGCUC-3′; SEQ ID NO: 28) encoding the influenza virus epitope GILGFVFTL (SEQ ID NO:30) preceded by a methionine (FIG. 7B), and transfection with the RNA Oligo CMV pp65 (5-OH AUGAACCUGGUGCCCAUGGUGGCUACGGUU-3′; SEQ ID NO: 31) encoding the CMV pp65 epitope MNLVPMVATV (SEQ ID NO: 32) preceded by a methionine (FIG. 7C). FIG. 7D shows the gating strategy on lymphocytes in forward scattering and side scattering and on CD3+ and CD4+ population in the case of the control culture with no RNA transfection. FIG. 7E shows the dot plot analysis of the no RNA culture after gating, FIG. 7F shows the dot plot analysis of the culture transfected with Oligo Flu matrix RNA after gating, and FIG. 7G shows the dot plot analysis of the culture transfected with the Oligo CMV pp65 RNA after gating.

FIG. 8: shows a graphical representation of IL-2 release by OT1 mouse splenocytes transfected with the indicated agents after 40 hours of incubation as measured in the cell supernatant. The reagents were as follows: SIINFEKL-CF: 5′-AUGAGUAUAAU[2′-F-C]AA[2′-F-C]UUUGAAAAA[2′-F-C]UG-3′ (wherein 2′-F-C denotes 2′-fluoro-deoxy-cytosine; SEQ ID NO: 25); SIINFEKL: 5′-AUGAGUAUAAUCAACUUUGAAAAACUG-3′ (SEQ ID NO: 25).

### DETAILED DESCRIPTION OF THE INVENTION

The present invention is further illustrated by the following non-limiting Examples. The following detailed description is merely exemplary in nature and is not intended to limit the compositions and systems contemplated herein, the methods for forming the hydrogel or the applicant and uses of the composition and systems. Furthermore, there is no intention to be bound by any theory presenting in the preceding background or following detailed description.

### EXAMPLES

### Example 1

The following RNA oligonucleotide was chemically synthesized using routine oligonucleotide synthesis by a commercial supplier (Bio-Synthesis, Inc., Lewisville, Tex., USA):

(SEQ ID NO: 22; start and stop codons
are shown underlined)
5′-ACAAGAUGGAGAGUAUAAUCAACUUUGAAAAACUGUAA-3′

A 5′-cap was generated chemically based on the method of Sekine, et al. (1996) J. Org. Chem. 61, 4412-4422, resulting in the following structure (start and stop codon are shown in underlined):

(SEQ ID NO: 22)
5′-N7-MeGppp-ACAAGAUGGAGAGUAUAAUCAACUUUGAAAAACUG
UAA-3′
(“SIINFEKL ChemRNA”)

The coding sequence encodes the amino acid sequence MESIINFEKL containing the epitope SIINFEKL of ovalbumin (positions 257 to 264 in ovalbumin; UniProt Acc. No. P01012).

In an alternative embodiment, a fully chemically synthesized RNA with the same sequence as above was prepared, but having a 3′-(A)₂₀ tail (again, start and stop codon are shown in capital letters):

(SEQ ID NO: 35)
5′ N7-MeGppp-ACAAGAUGGAGAGUAUAAUCAACUUUGAAAAACUG
UAAAAAAAAAAAAAAAAAAAAAA-3′

In a comparative example (“SIINFEKL ChemRNA Poly-A”), the above RNA without poly(A) tail (SIINFEKL ChemRNA) was polyadenylated by incubation for 2 hours with poly-A-polymerase in the presence of ATP using a commercially available enzyme (E. coli Poly(A) Polymerase, catalogue no. M0276, New England Biolabs Inc., Ipswich, Mass., USA) according to the manufacturer's instructions.

As controls, the following RNAs were used:

Positive control: enzymatically prepared mRNA coding for ovalbumin (Trilink Biotechnologies, LLC, San Diego, Calif., USA).
Negative control: enzymatically prepared mRNA coding for luciferase (prepared in the laboratory of the inventor).

RNA was formulated with the lipofectamine reagent MessengerMax (Thermo Fischer Scientific Corp., Waltham Mass., USA) by mixing 200 ng of RNA and 400 ng MessengerMax or 20 ng of RNA and 40 ng MessengerMax or 2 ng of RNA and 4 ng MessengerMax per cell culture well. The mixture was transfected into RAG2 KO C57Bl/6 mouse OT1 splenocytes alone and said splenocytes plus syngenic B16 tumor cells, respectively, by adding 100,000 splenocytes in 100 μl medium per well (for splenocytes alone) or by adding 100,000 splenocytes in 100 μl plus 50,000 B16 cells in 100 μl per well (for splenocytes plus B16 cells) according to the manufacturer's instructions for the MessengerMax reagent.

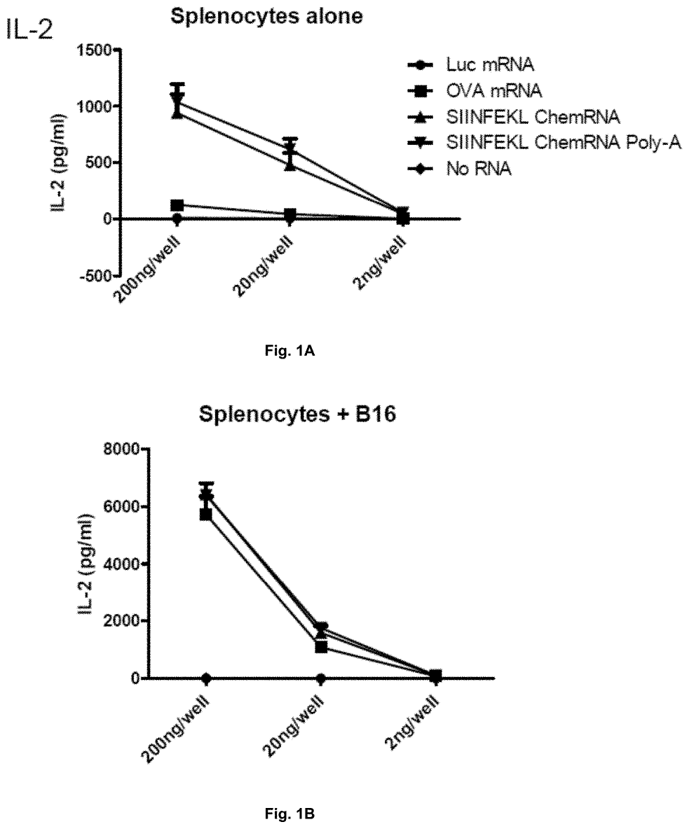

After incubation for 18 hours, IFN-gamma and IL-2, respectively was measured by ELISA (biological triplicates) in the culture supernatants using commercially available assays (ELISA MAX™ Standard Set Mouse IFN-γ and ELISA MAX™ Standard Set Mouse IL-2, both from BioLegend Inc., San Diego, Calif., USA). The cytokines are produced by OT1 cells and released in the culture medium when the T-lymphocytes are activated, i.e. when they recognize the SIINFEKL peptide on the H-2 Kb mouse class I molecule. The results are shown in FIG. 1 (IL-2 release; A: splenocytes alone; B splenocytes plus B16 cells) and 2 (IFN-gamma release; A: splenocytes alone; B splenocytes plus B16 cells).

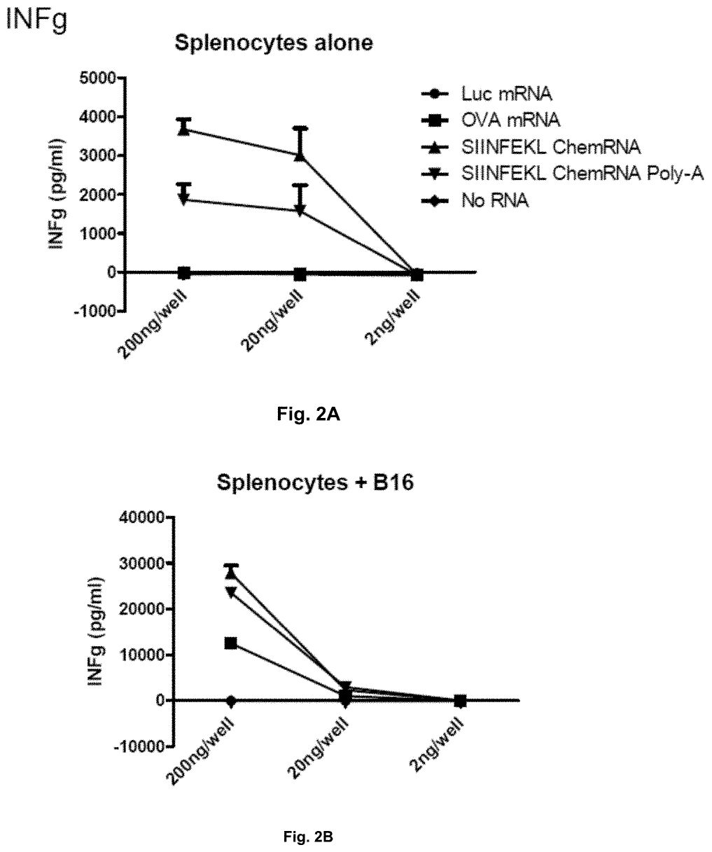

FIGS. 1 and 2 show that the fully chemically synthesized RNA SIINFEKL ChemRNA generated strong release of IL-2 and IFN-gamma, respectively, by OT1 cells. The signal produced is stronger than in the case of the positive control OVA mRNA (enzymatically synthesized mRNA coding for full length ovalbumin). Treatment of SIINFEKL ChemRNA by a poly-A polymerase does not improve the efficacy of the chemically synthesized oligonucleotide. It is furthermore highly surprising that the poly(A) tail is not required to generate a strong cytokine response.

### Example 2

The following RNAs were prepared by chemical synthesis by commercial suppliers (Bio-Synthesis, Inc., Lewisville, Tex., USA, or Microsynth AG, Balgach, Switzerland, respectively) and, where required, capped as outlined in Example 1:

In the following sequences, capital letters indicate start and stop codons, respectively. The constructs contain a 5′UTR sequence (acaag) directly preceding the start codon.

(a) Capped 5n SIINFEKL: 5′-N7-MeGppp, 7mGppp-acaagAUGaguauaaucaacuuugaaaaacugUAA-3′ (encoding the SIINFEKL epitope as described above in Example 1).
This construct thus comprises a 5′-Cap structure, but no poly(A) tail (see also Example 1).
(b) ppp 5n SIINFEKL: 5′-ppp-acaagAUGaguauaaucaacuuugaaaaacugUAA-3′ (SEQ ID NO: 22; encoding the SIINFEKL epitope as described above in Example 1).
This construct lacks a 5′-Cap structure and a poly(A) tail. The construct has a triphosphate group at the 5′ end (denoted 5′-ppp).
(c) p 5n SIINFEKL: 5′-p-acaagAUGaguauaaucaacuuugaaaaacugUAA-3′ (SEQ ID NO: 22; encoding the SIINFEKL epitope as described above in Example 1).
This construct lacks a 5′-Cap structure and a poly(A) tail. The construct has a monophosphate group at the 5′ end (denoted 5′-p).
(d) OH 5n SIINFEKL: 5′-OH-acaagAUGaguauaaucaacuuugaaaaacugUAA-3′ (SEQ ID NO: 22; encoding the SIINFEKL epitope as described above in Example 1).
This construct lacks a 5′-Cap, and has also not even phosphate at the C-5′ of the 5′ terminal ribose which therefore carries only 5′-OH group. Furthermore, the construct lacks a poly(A) tail.
(e) Kif18b capped oligo: 5′-N7-MeGppp, 7mGppp-acaagAUGuuccaggaauuuguugacugggaaaacguuUAA-3′ (SEQ ID NO: 23; encoding MFQEFVDWENV (SEQ ID NO: 34) of mutated anti-kinesin family member 18b).
The oligonucleotide serves as a negative control here.

Ovalbumin mRNA served as a positive control. Splenocytes alone with no transfection of any RNA served as a further negative control.

Mouse OT1 splenocytes (100,000 cells in 100 μl per well) were transfected with the above oligonucleotides as described in Example 1, except that 200 ng, 20 ng, and 5 ng, respectively, of RNA were used per well.

In a further experiment, the RNAs (a), (c) and (e) were polyadenylated as described in Example 1 and then used for transfection in the amounts (as of ChemRNA, not taking into account additional weight from the added poly(A) tail) as outlined above.

The cells were incubated for 18 hours and IL-2 was measured in the culture supernatant as described in Example 1.

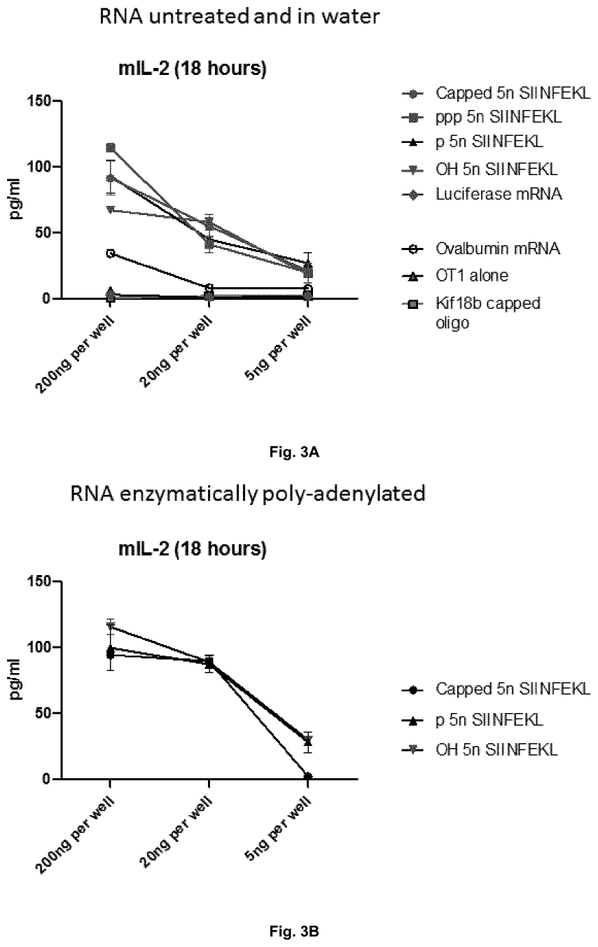

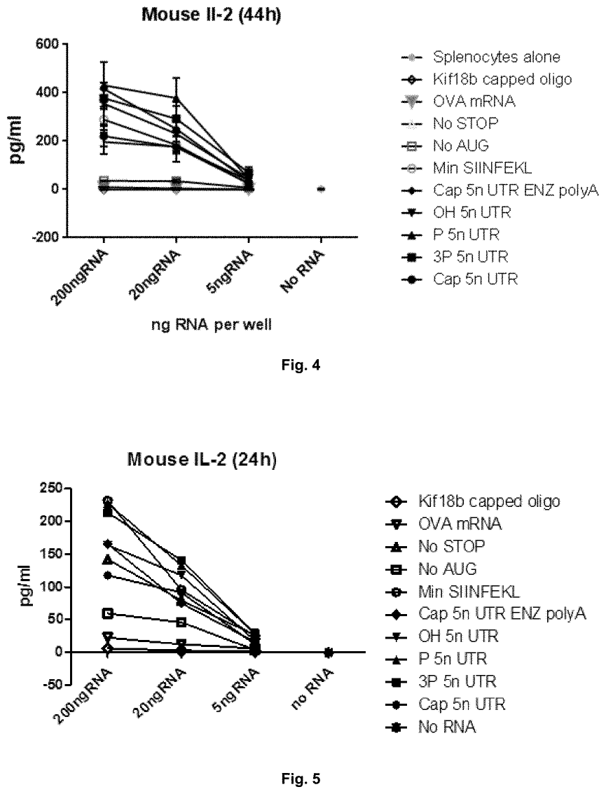

The results are shown in FIGS. 3A (no polyadenylated RNA) and 3B (polyadenylated constructs (a), (c) and (e)).

Very surprisingly, all constructs (a) to (d) exerted a higher release of IL-2 by the OT1 splenocytes in comparison to the full length ovalbumin mRNA. Thus, even if the RNA of the invention lacks a 5′-Cap and a poly (A) tail it is expressed in the splenocytes and results in strong IL-2 expression. Even more surprisingly, this is the case when the RNA has only a 5′-triphosphate or even no phosphorylation at the 5′-terminal nucleotide. As shown in FIG. 3B, addition of a short poly (A) tail provides for a slightly higher IL-2 at lower amounts of the tested constructs in comparison to the respective non-polyadenylated RNA.

### Example 3

In order to further elucidate whether RNAs having even less structural requirements that are normally attributed to mRNAs for eliciting an IL-2 response in immune cells the further constructs were synthesized by a commercial supplier (Microsynth AG, Balgach, Switzerland):

(f) Min SIINFEKL: 5′-AUGAGUAUAAUCAACUUUGAAAAACUG-3′ (SEQ ID NO: 25; encoding the SIINFEKL epitope as described above in Example 1).
This construct lacks, in addition to a Cap or phosphate group at the 5′ terminus (which is OH) and a poly(A) tail, also a 5′UTR sequence and it even has no stop codon.
(g) No STOP: 5′-ACAAGAUGAGUAUAAUCAACUUUGAAAAAC UG-3′ (SEQ ID NO: 26; encoding the SIINFEKL epitope as described above in Example 1).
This construct corresponds to construct (6) except that it contains the 5′UTR ACAAG.
(h) No AUG: 5′-AGUAUAAUCAACUUUGAAAAACUG-3′ (SEQ ID NO: 27; encoding the SIINFEKL epitope as described above in Example 1).
This construct (h) is the absolute minimal ChemRNA, since it consists of the coding sequence of the indicated epitope only (at the 5′ end it has no Cap structure or phosphate groups, and thus the group attached to the 5′-C of the 5′-terminal nucleotide is OH). It has no canonical start codon.

Constructs (a) to (h) were transfected into OT1 mouse splenocytes using the method as outlined in Example 1, but employing 200 ng, 20 ng or 5 ng RNA. After incubation for 44 h the IL-2 level was measured in the culture supernatants as outlined Example 1. The results are shown in FIG. 4. Completely unexpectedly, also constructs (f), (g) and even (h) (i.e. the coding sequence without start codon) resulted in an IL-2 release by the OT1 cells clearly higher than the negative controls and even higher than the positive control: the ovalbumin mRNA.

The experiments were repeated, but with measurement of IL-2 after 24 h, and the results are shown in FIG. 5, confirming the results obtained after incubation for 44 h. Interestingly, construct (f) (characterized by only having a start codon but no other attributes of a bone fide mRNA) gave the highest IL-2 concentration at 200 ng after 24 h of incubation.

### Example 4

It was further investigated whether RNAs of the present invention used for expression of peptides, especially oligopeptides such as, preferably epitopes of infectious agents and of cancer peptides, also provide for expression of such peptides in human cells.

As exemplary constructs for expression of viral peptides the following constructs were prepared by a commercial supplier (Microsynth AG, Balgach, Switzerland) using chemical synthesis:

(i) Oligo Flu matrix: 5-OH-AUGGGGAUUUUGGGGUUUGUGUUCACGCUC-3′ (SEQ ID NO: 28), encoding MGILGFVFTL (SEQ ID NO: 29), i.e. the influenza virus M peptide GILGFVFTL (SEQ ID NO: 30) which is commonly used to stimulate human Influenza-specific CD8+ T-cells.
(j) Oligo CM pp65: 5′-OH AUGAACCUGGUGCCCAUGGUGGCUACGGUU-3′ (SEQ ID NO: 31), encoding MNLVPMVATV (SEQ ID NO: 32), i.e. the cytomegalo virus (CMV) pp65 peptide (NLVPMVATV; SEQ ID NO: 33) commonly used for stimulation of human CMV-specific CD8+|T-cells.

Both constructs consist only of a start codon (no Cap and no phosphate group(s) at the 5′ terminus) and the coding sequence (no stop codon, no poly (A) tail).

PBMCs were isolated from the blood of a healthy voluntary HLA-A2 positive donor. 10 million of PBMCs were used for starting three cultures in 10 ml complete medium each. For transfection RNA was formulated with the lipofectamine reagent MessengerMax (Thermo Fischer Scientific Corp., Waltham Mass., USA) by mixing 1 μg of RNA in 25 μl OptiMEM medium and 2 μg MessengerMax in 25 μl OptiMEM medium. Each mixture was added to the respective PBMC culture. The third culture was treated with the same 50 μl mixture which had no RNA added. After one week of incubation antibody and tetramer staining were carried on 2 ml of cell culture. FACS analyses were carried out with the following settings:

FACS: gate on lymphocytes in FSC-SSC
Phycoerithryn (PE): FLU Matrix tetramer (HLA-A2 with peptide: GILGFVFTL; SEQ ID NO: 30)
Allophycocyanin (APC): CMV pp65 tetramer (HLA-A2 with peptide: NLVPMVATV; SEQ ID NO: 33).

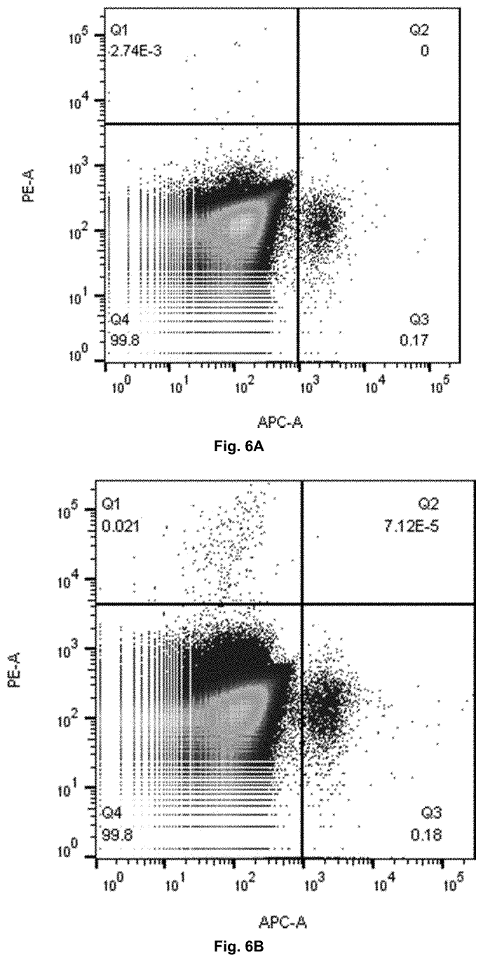

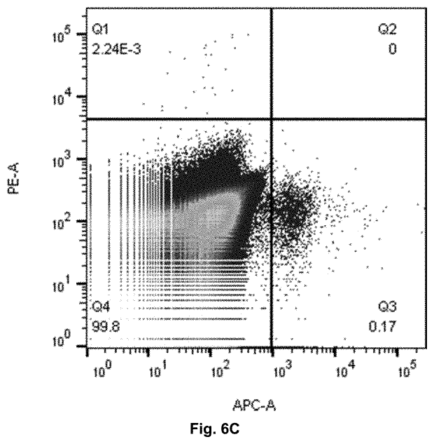

The results are shown in FIG. 6.

The experiment was prolonged for a further week to a total of 2 weeks of cell culture before FACS analyses wherein at day 7 the transfection protocol was repeated and the medium replaced by fresh medium supplemented with 5 ng/ml recombinant human IL-2. The replacement with fresh medium supplemented with 5 ng/ml recombinant human IL-2 was repeated at days 9 and 12.

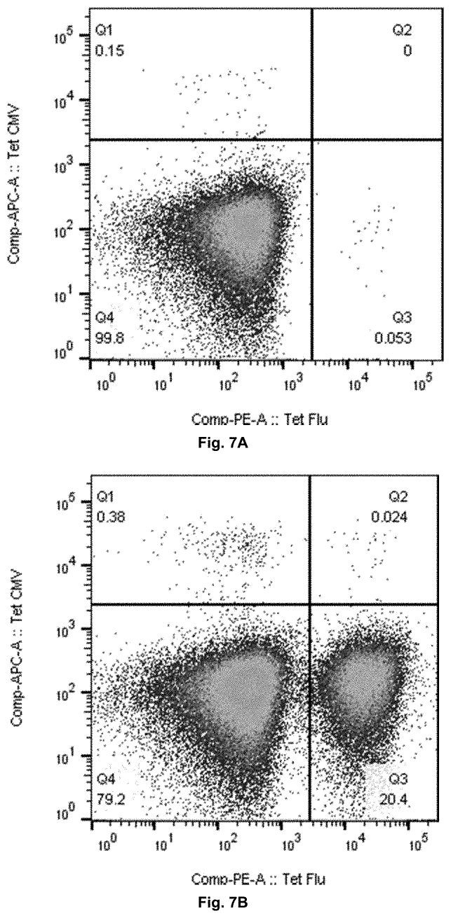

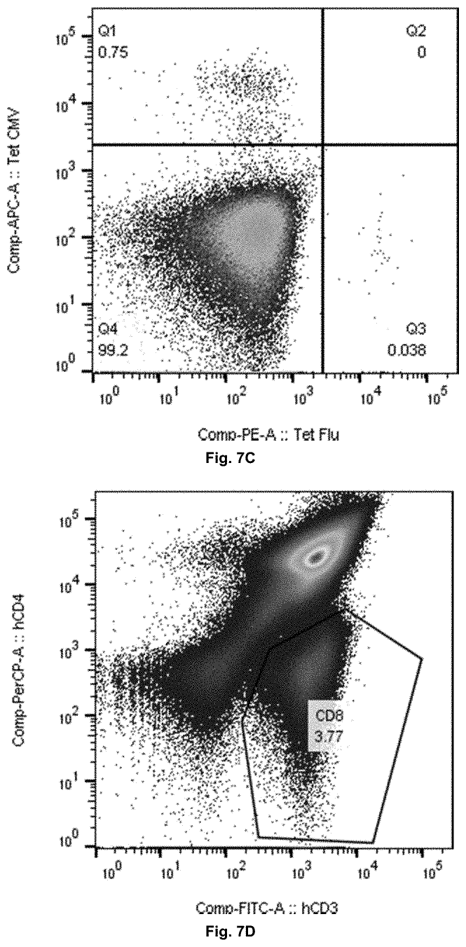

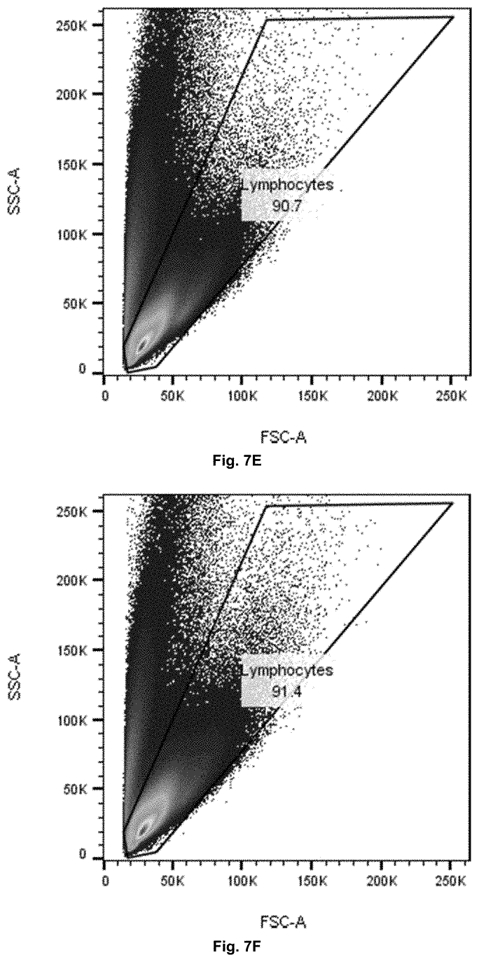

The results are shown in FIG. 7.

The experiments demonstrate that uncapped ChemRNAs of the invention having no 5′-Cap structure, having not 5′-phosphate(s), lacking a 5′UTR, lacking a stop codon and also a poly(A) tail, are expressed by PBMCs and presented to human T cells:

The FACS analysis shown in FIG. 6B (compared with the culture with no transfection of RNA) demonstrates that, after one week, clearly in the PBMC culture transfected with RNA coding the FLU epitope, FLU-specific T-cells have proliferated. This demonstrates that the RNA-encoded epitope was produced and presented to the T cells.

After two weeks, the signal in the culture transfected with Oligo Flu matrix substantially increased (FIG. 7B), and also a positive signal was detected in the culture transfected with Oligo CMV pp65 RNA (FIG. 7 C). The signal for the latter is weaker in comparison to the culture transfected with the Oligo Flu matrix RNA, which can either due to the cells being anergic or a non-optimal peptide sequence.

### Example 5

It was further investigated whether RNAs of the present invention used for expression of peptides, especially oligopeptides such as, preferably epitopes of infectious agents and of cancer peptides, also provide for expression of such peptides, in case modified nucleotides are included in the RNA sequence, in particular in the coding sequence.

The constructs used were as follows:

SIINFEKL: 5′-AUGAGUAUAAUCAACUUUGAAAAACUG-3′ (SEQ ID NO: 25; encoding the SIINFEKL epitope as described above in Example 1). This constructs corresponds to construct (f) of Example 2.
SIINFEKL CF: 5′-AUGAGUAUAAU[2′-F-C] AA[2′-F-C]UUUGAAAAA[2′-F-C]UG-3′ (SEQ ID NO: 25; encoding the SIINFEKL epitope as described above in Example 1), wherein [2′-F-C] denotes 2′-fluoro-deoxycytosine).

As controls, the following RNAs were used:

Positive control: enzymatically prepared mRNA coding for ovalbumin (Trilink Biotechnologies, LLC, San Diego, Calif., USA).
Negative control: enzymatically prepared mRNA coding for luciferase (prepared in the laboratory of the inventor).

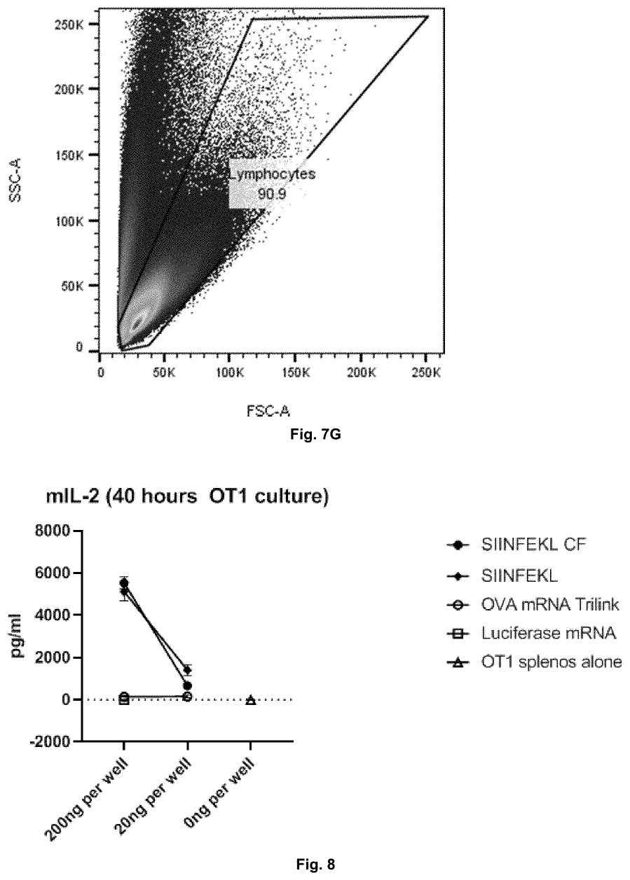

Mouse OT1 splenocytes (100,000 cells in 100 μl per well) were transfected with the above oligonucleotides as described in Example 1, except that 200 ng, 20 ng, and (as a negative control) 0 ng, respectively, of RNA were used per well. After incubation for 40 h the IL-2 level was measured in the culture supernatants as outlined Example 1. The results are shown in FIG. 8.

As demonstrated in FIG. 8, the inclusion of 2-F-C into the RNA does not interfere with the expression of the encoded peptide.

The present invention shows that various chemically synthesized RNA species having a coding sequence, preferably encoding a peptide, more preferably, an epitope such as an epitope of an infectious agent such as a virus, or an epitope of a tumor antigen, but lacking one, multiple or even any of messenger RNA-specific structural features can be expressed by living cells, specifically mammalian cells, which is a completely unexpected finding, and opens up the use of minimal ChemRNAs for various diagnostic and therapeutic applications.

First of at all, it is unexpected that a 5′-capped RNA having an AUG start codon in a non-Kozak (TISU) surrounding and lacking a poly (A) tail showed stimulation of specific T-cells (IL-2 production in transfected mouse OT1 splenocytes). The further investigations according to the invention lead to even more surprising results: no cap is factually necessary for stimulation of OT1 mouse splenocytes to release IL-2. It is even possible to use RNAs having no 5′ phosphate(s) but having a 5′-OH group instead. The further results of the present invention show that a 5′UTR is not an absolute requirement for the expression of the coding sequence. Although the presence of a start codon is preferred for optimizing the expression of the coding sequence, a start codon is also not an absolute requirement. A stop codon needs not to be present either, if the last nucleotide on the 3′ end of the RNA is the last nucleotide of the coding sequence.

1. A fully chemically synthesized RNA having the structure according to the following general formula (1):
5′-W-X-Y-(coding sequence)-Z-3′ (1)
wherein
W is selected from the group consisting of a 5′-Cap, a free 5′-triphosphate group, a free 5′-disphosphate group, a free 5′-diphosphate group, a free 5′-monophosphate group, a free 5′-OH group and chemically modified analogues of said 5′-Cap, said 5′-triphosphate group, said free 5′-disphosphate group or said free 5′-monophosphate group;
X may or may not be present, and, if present is a 5′UTR sequence;
Y may or may not be present, and, if present is a start codon; and
Z is directly linked to the coding sequence and is selected from the group consisting of a free 3′-OH group, a stop codon and a stop codon linked, optionally via a 3′UTR sequence, to a poly(A) tail.

2. The RNA of claim 1 having a structure selected from the group consisting of the following general formulas (2) to (61):
5′-N7MeGppp-UTR-AUG-(coding sequence)-stop-polyA-3′ (2)
5′-N7MeGppp-UTR-AUG-(coding sequence)-stop-3′ (3)
5′-N7MeGppp-UTR-AUG-(coding sequence)-3′ (4)
5′-N7MeGppp-UTR-(coding sequence)-stop-polyA-3′ (5)
5′-N7MeGppp-UTR-(coding sequence)-stop-3′ (6)
5′-N7MeGppp-UTR-(coding sequence)-3′ (7)
5′-N7MeGppp-AUG-(coding sequence)-stop-polyA-3′ (8)
5′-N7MeGppp-AUG-(coding sequence)-stop-3′ (9)
5′-N7MeGppp-AUG-(coding sequence)-3′ (10)
5′-N7MeGppp-(coding sequence)-stop-polyA-3′ (11)
5′-N7MeGppp-(coding sequence)-stop-3′ (12)
5′-N7MeGppp-(coding sequence)-3′ (13)
5′-triP-UTR-AUG-(coding sequence)-stop-polyA-3′ (14)
5′-triP-UTR-AUG-(coding sequence)-stop-3′ (15)
5′-triP-UTR-AUG-(coding sequence)-3′ (16)
5′-triP-UTR-(coding sequence)-stop-polyA-3′ (17)
5′-triP-UTR-(coding sequence)-stop-3′ (18)
5′-triP-UTR-(coding sequence)-3′ (19)
5′-triP-AUG-(coding sequence)-stop-polyA-3′ (20)
5′-triP-AUG-(coding sequence)-stop-3′ (21)
5′-triP-AUG-(coding sequence)-3′ (22)
5′-triP-(coding sequence)-stop-polyA-3′ (23)
5′-triP-(coding sequence)-stop-3′ (24)
5′-triP-(coding sequence)-3′ (25)
5′-diP-UTR-AUG-(coding sequence)-stop-polyA-3′ (26)
5′-diP-UTR-AUG-(coding sequence)-stop-3′ (27)
5′-diP-UTR-AUG-(coding sequence)-3′ (28)
5′-diP-UTR-(coding sequence)-stop-polyA-3′ (29)
5′-diP-UTR-(coding sequence)-stop-3′ (30)
5′-diP-UTR-(coding sequence)-3′ (31)
5′-diP-AUG-(coding sequence)-stop-polyA-3′ (32)
5′-diP-AUG-(coding sequence)-stop-3′ (33)
5′-diP-AUG-(coding sequence)-3′ (34)
5′-diP-(coding sequence)-stop-polyA-3′ (35)
5′-diP-(coding sequence)-stop-3′ (36)
5′-diP-(coding sequence)-3′ (37)
5′-mP-UTR-AUG-(coding sequence)-stop-polyA-3′ (38)
5′-mP-UTR-AUG-(coding sequence)-stop-3′ (39)
5′-mP-UTR-AUG-(coding sequence)-3′ (40)
5′-mP-UTR-(coding sequence)-stop-polyA-3′ (41)
5′-mP-UTR-(coding sequence)-stop-3′ (42)
5′-mP-UTR-(coding sequence)-3′ (43)
5′-mP-AUG-(coding sequence)-stop-polyA-3′ (44)
5′-mP-AUG-(coding sequence)-stop-3′ (45)
5′-mP-AUG-(coding sequence)-3′ (46)
5′-mP-(coding sequence)-stop-polyA-3′ (47)
5′-mP-(coding sequence)-stop-3′ (48)
5′-mP-(coding sequence)-3′ (49)
5′-OH-UTR-AUG-(coding sequence)-stop-polyA-3′ (50)
5′-OH-UTR-AUG-(coding sequence)-stop-3′ (51)
5′-OH-UTR-AUG-(coding sequence)-3′ (52)
5′-OH-UTR-(coding sequence)-stop-polyA-3′ (53)
5′-OH-UTR-(coding sequence)-stop-3′ (54)
5′-OH-UTR-(coding sequence)-3′ (55)
5′-OH-AUG-(coding sequence)-stop-polyA-3′ (56)
5′-OH-AUG-(coding sequence)-stop-3′ (57)
5′-OH-AUG-(coding sequence)-3′ (58)
5′-OH-(coding sequence)-stop-polyA-3′ (59)
5′-OH-(coding sequence)-stop-3′ (60)
5′-OH-(coding sequence)-3′ (61)
wherein:
polyA is a poly(A) tail;
stop is a stop codon;
UTR is a 5′UTR
triP is a free triphosphate group;
diP is a free diphosphate group;
mP is a free monophosphate group.

3. The RNA of claim 2 having a structure selected from the group consisting of formulas (3), (15), (39), (51) and (58).

4. The RNA according to claim 2 wherein, if present, the UTR has a length of from 2 to 10 nt.

5. The RNA of claim 4 wherein the UTR has a length of from 2 to 5 nt.

6. The RNA of claim 4 wherein the UTR comprises the sequence aag.

7. The RNA of claim 6 wherein the UTR consists of the sequence acaag.

8. The RNA according to claim 2 wherein, if present, the poly(A) tail has a length of up to 30 nt, preferably 5 to 30 nt.

9. The RNA of claim 8 wherein the poly(A) tail has a length of up to 20 nt, preferably 5 to 20 nt.

10. The RNA of claim 9 wherein the poly(A) tail has a length of up to 10 nt, preferably 5 to 10 nt.

11. The RNA according to claim 2 wherein the coding sequence encodes an amino acid sequence of up 65 amino acids, preferably of from 4 to 40 amino acids.

12. The RNA of claim 11 wherein the coding sequence encodes 4 to 30 amino acids, preferably 8 to 20 amino acids.

13. The RNA according to claim 2 wherein the RNA is a RNA oligonucleotide.

14. The RNA of claim 13 consisting of at most 200 nt, preferably at most 120 nt, more preferably of at most 100 nt.

15. The RNA according to claim 1 comprising at least one chemical modification, preferably at a single nucleotide, more preferably at a terminal nucleotide.

16. The RNA according to claim 1 wherein the RNA is modified enzymatically.

17. The RNA according to claim 1 wherein the coding sequence encodes a peptide of an infectious agent or a cancer peptide or a peptide of a tissue recognized by autoimmune cells.

18. The RNA of claim 17 wherein the infectious agent is selected from viruses, bacteria and fungi.

19. The RNA of claim 17 wherein the cancer peptide has an amino acid sequence which comprises at least one amino acid that is different from the amino acid sequence of the non-cancer wildtype.

20. The RNA according to claim 1 wherein the coding sequence of the RNA is expressed when the RNA is present in a cell or an organism or a cell-free expression system.

21. The RNA according to claim 1 wherein the RNA is single stranded.

22. A RNA population wherein at least 85%, preferably at least 90%, more preferably at least 95% of the RNAs in said population are the RNA according to claim 1.

23. A RNA population comprising the RNA according to claim 1 and having a full length of n nt and at least 1% of a RNA having a chemical composition being at least 95%, preferably at least 96%, more preferably at least 97%, still more preferred at least 98%, even more preferred at least 99% identical to the chemical composition of the RNA but having a length of (n−1) nt wherein the percentage of identity of the chemical composition of the RNA of length (n−1) to the chemical composition of the RNA of length n is based on the chemical composition of the (n−1) nucleotides of the full length RNA of length n.

24. The RNA population of claim 22 comprising at least 1% of a RNA having a chemical composition being at least 93%, preferably at least 95%, more preferably at least 97%, still more preferred at least 98%, even more preferred at least 99% identical to the chemical composition of the full length RNA as the full length RNA but having a length of (n−2) wherein the percentage of identity of the chemical composition of the RNA of length (n−2) to the chemical composition of the full length RNA of length is meant with respect to the chemical composition of the (n−2) nucleotides of the full length RNA of length n.

25. The RNA population according to claim 22 comprising at least 1% of a RNA having a chemical composition being at least 90%, preferably at least 95%, more preferably at least 97%, still more preferred at least 98%, even more preferred at least 98.5% identical to the chemical composition of the full length RNA as the full length RNA but having a length of (n−3) wherein the percentage of identity of the chemical composition of the RNA of length (n−3) to the chemical composition of the full length RNA of length is meant with respect to the chemical composition of the (n−3) nucleotides of the full length RNA of length n.

26. A pharmaceutical composition comprising the RNA according to claim 1.

27. A diagnostic kit comprising at least one RNA according to claim 1.

28. The kit of claim 27 wherein the coding sequence encodes a peptide of an infectious agent.

29. The kit of claim 28 further comprising T cells specific for said infections agent.

30. A vaccine comprising the RNA according to claim 1.

31. The RNA according to claim 1 for use as a medicament.

32. Use of the RNA according to claim 1 for diagnostic purposes.

33. Use of the RNA according to claim 1 for expressing the coding sequence in a cell-free expression system, a cell or an organism.

34. A method for expressing an amino acid sequence in a cell or organism comprising the step of introducing the RNA according to claim 1 into the cell or organism.

35. A method for expressing an amino acid sequence in a cell-free expression system comprising the step of incubating the RNA according to claim 1 in the presence of the cell-free expression system.

36. The RNA according to claim 1 for use in the treatment of cancer and tumors.

37. The RNA for the use of claim 36 wherein the treatment comprises vaccination of a cancer patient against the cancer the patient is suffering from.

38. The RNA according to claim 19 for use in the treatment of cancer and tumors.

39. The RNA according to claim 1 for use in the treatment and/or prevention of infectious diseases.
